(19) United States
(12) Patent Application Publication
(10) Pub. No.: US 2017/0246421 A1
(43) Pub. Date: Aug. 31, 2017

(54) RESPIRATION VALVE AND BREATHING MACHINE HAVING SAME

(71) Applicant: BEIJING AEONMED CO., LTD., Beijing (CN)

(72) Inventor: Jinquan Ji, Beijing (CN)

(21) Appl. No.: 15/120,992

(22) PCT Filed: Dec. 31, 2014
(86) PCT No.: PCT/CN2014/095775

§ 371 Date: Aug. 23, 2016

(30) Foreign Application Priority Data
Feb. 28, 2014 (CN) 20140072610.2

(51) Int. Cl. A61M 16/20; A61M 16/08

(52) U.S. Cl. CPC A61M 16/206; A61M 16/0866

(57) ABSTRACT

A respiration valve (100) and breathing machine having same; the respiration valve (100) comprises a valve body (1), an air resistor (2), a stainless steel valve port (3), a diaphragm (4) and valve bonnet (5); the valve body (1) is formed with an air intake channel (101) and an air output channel (102) connected to the air intake channel (101), and the air intake channel (101) has an air inlet (103) and an air outlet (104); the air resistor (2) is disposed in the air intake channel (101); the stainless steel valve port (3) is provided at the air outlet (104) of the air intake channel of the valve body (1), and one end of the stainless steel valve port (3) extends out of the air outlet (104) of the air intake channel of the valve body (1); the diaphragm (4) is movably disposed on the valve body (1) between the open and close positions of the stainless steel valve port (3); the valve bonnet (5) is disposed on the valve body (1), and cooperates with the valve body (1) to press against the edge of the diaphragm (4); and the valve bonnet (5) is provided with a through hole (501) thereon. The respiration valve (100) improves the sealing property of the diaphragm (4) and the stainless steel valve port (3), ensures accurate and reliable opening and closing of the valve port (3), and can precisely calculate the moisture volume inhaled and exhaled by the respiration valve (100).

The present invention application claims the priority of Chinese Patent Application No. 201410072610.2, titled “RESPIRATION VALVE AND BREATHING MACHINE HAVING SAME”, filed by Beijing Aeonmed Co., Ltd. on Feb. 28, 2014, which is incorporated herein by reference in its entirety.

### TECHNICAL FIELD

The present invention relates to a technical field of medical equipment manufacturing, in particular to a respiration valve and breathing machine having the respiration valve.

### BACKGROUND OF THE INVENTION

A respiration valve in related technologies is made entirely from aluminum alloy, a reliable sealing is required at a diaphragm and a sealing valve port of the respiration valve, freely opening and closing thereof is also required. The processing precision is very high. There are certain difficulties to keep the valve port with a low roughness due to the characteristics of the aluminum alloy, thus the sealing performance at the diaphragm and the sealing valve port of the respiration valve is always not so good, in this way resulting in distortion in monitoring and calculating of a flow sensor. Furthermore, if the valve port is scratched accidentally in the process of machining, transportation and installation, the entire respiration valve will be scrapped, being not beneficial for saving and maintaining. In addition, a sealing mandrel of a plastic steam trap and a tray of a waterlogging cup will be damaged during a high temperature disinfection process.

### SUMMARY OF THE INVENTION

The present invention aims to solve one of the above described technical problems in prior art at least to a certain extent. Therefore, an object of a first aspect of the present invention is to provide a respiration valve, which has a good sealing performance, and can reduce the risk of air leakage.

An object of a second aspect of the present invention is to provide a breathing machine having the respiration valve.

A respiration valve according to an embodiment of the first aspect of the present invention comprises a valve body, an air resistor, a stainless steel valve port, a diaphragm and a valve bonnet. An air intake channel and an air output channel capable of being in communication with the air intake channel are formed within the valve body, and the air intake channel has an air inlet and an air outlet; the air resistor is disposed within the air intake channel; the stainless steel valve port is provided at the air outlet of the valve body, and one end of the stainless steel valve port extends out of the air outlet of the valve body; the diaphragm is movably disposed on the valve body between opened and closed positions of the stainless steel valve port; the valve bonnet is disposed on the valve body, and cooperates with the valve body to press against an edge of the diaphragm, the valve bonnet is provided with a through hole thereon.

With the respiration valve according to the embodiment of the present invention, the performance of the sealing of the diaphragm and the stainless steel valve port is improved, an accurate and reliable opening and closing of the valve port in normal working environment is realized, and the tidal volume inhaled and exhaled by the respiration valve may be precisely calculated, the patient may be given with a comfortable breathing mode.

Furthermore, the respiration valve according to the above described embodiment of the present invention may further comprise the following additional technical features:

according to an embodiment of the present invention, a joint between the air intake channel and the air output channel is formed with a step surface, a part of the stainless steel valve port is disposed within the air intake channel, and the other part of the stainless steel valve port extends out of the air outlet, a limiting embossment is formed on an outer wall surface of the part of the stainless steel valve port which extends out of the air outlet, and the limiting embossment abuts against the step surface. Thus it can prevent the stainless steel valve port from moving into the air intake channel under the abutment action of the diaphragm.

According to an embodiment of the present invention, the air resistor is an aluminium alloy air resistor or a stainless steel air resistor, the valve body is an alumunium alloy valve body or a stainless steel valve body. Thus the respiration valve has a high-strength structure, facilitates to be produced and assembled, and is rust protective.

According to an embodiment of the present invention, the air resistor is formed integrally with the stainless steel valve port. Thus the assembly elements of the respiration valve are decreased, simplifying the assembling process of the respiration valve, and improving the assembly efficiency of the respiration valve.

According to an embodiment of the present invention, a seal ring is provided between an outer wall surface of the stainless steel valve port and an inner wall surface of the air intake channel. Thus it facilitates the sealing between the stainless steel valve port and the inner wall of the air intake channel, thereby avoiding air leakage.

According to an embodiment of the present invention, a limiting groove is provided on an outer wall of the air resistor, and a jackscrew with an end fitted in the limiting groove is provided on the valve body. Thus the air resistor can be located to avoid the sliding thereof.

According to an embodiment of the present invention, a bottom of the valve body is provided with a logged water outlet in communication with the air intake channel, and a waterlogging cup component in communication with the logged water outlet is provided on the valve body, the waterlogging cup component comprises: a waterlogging cup connector, a waterlogging cup, a seal ball and an ejector rod. An upper end of the waterlogging cup connector is connected with valve body and in communication with the logged water outlet, and an inner wall of the waterlogging cup component is provided with an annular flange; the waterlogging cup is detachably connected with an lower end of the waterlogging cup connector; the seal ball is movably disposed within the waterlogging cup connector between a first position and a second position, wherein the seal ball abuts against the annular flange to close the waterlogging cup connector at the first position, and moves upwards to depart from the first position to open the waterlogging cup connector at the second position; the ejector rod is capable of abutting against a bottom wall of the waterlogging cup to push the seal ball to the second position, one end of the ejector rod is connected with the seal ball, and the other end of the ejector rod extends into the waterlogging cup. Thus it is easy to clean the water in the air intake channel, facilitates the use of the respiration valve.

According to an embodiment of the present invention, the seal ball is a high temperature resistance seal ball. Thus, the high temperature resistance seal ball will not be damaged and deformed when high temperature disinfection is performed on the respiration valve, such that the seal ball can seal the logged water outlet reliably, avoiding air leakage.

According to an embodiment of the present invention, a bottom surface of the valve body is provided with an annular notch surrounding the logged water outlet, the upper end of the waterlogging cup connector is fitted in the annular notch, and the waterlogging cup connector is connected with the valve body via a bolt directly. Thus it is easy to assemble the respiration valve, improving the assembly efficiency and production efficiency of the respiration valve.

A breathing machine according to an embodiment of the second aspect of the present invention comprises the respiration valve according to the first aspect of the present invention.

The breathing machine according to the embodiment of the present invention has the respiration valve according to the above described embodiments of the present invention. The performance of the sealing of the diaphragm and the stainless steel valve port is improved, the accurate and reliable opening and closing of the valve port under normal working environment is realized, and the tidal volume inhaled and exhaled by the respiration valve can be precisely calculated, the patient may be given with a comfortable breathing mode.

### BRIEF DESCRIPTION OF THE DRAWINGS

FIG. 1 is a schematic view of a respiration valve according to an embodiment of the present invention.

FIG. 2 is a schematic view of a respiration valve according to an embodiment of the present invention.

FIG. 3 is a schematic view of a valve body of a respiration valve according to an embodiment of the present invention.

Reference Numbers: respiration valve 100; valve body 1; air resistor 2; stainless steel valve port 3; diaphragm 4; valve bonnet 5; air intake channel 101; air output channel 102; air inlet 103; air outlet 104; step surface 105; limiting embossment 31; seal ring 6; jackscrew 7; waterlogging cup component 8; waterlogging cup connector 81; waterlogging cup 82; seal ball 83; ejector rod 84; annular flange 85; logged water outlet 106; annular notch 107.

### DETAILED DESCRIPTION OF THE INVENTION

Embodiments of the present invention will be described in detail below, examples of the embodiments are shown in the drawings, wherein same or similar reference numbers represent same or similar elements or elements with same or similar functions throughout the drawings. The embodiments described below with reference to the drawings are merely illustrative, intended to explain the present invention, and cannot be understood as a limiting to the present invention.

In the description of the present invention, it should be understood that, orientational or positional relationships indicated by terms of “center”, “longitudinal”, “lateral”, “length”, “width”, “thickness”, “up”, “down”, “front”, “back”, “left”, “right”, “vertical”, “horizontal”, “top”, “bottom”, “within”, “outside”, “clockwise”, “counterclockwise” and the like are orientational or positional relationships shown based on the drawings, merely to be convenient to describe the present and simplify the description, and not to express or imply that the indicated device or component must have a specific orientation, be constructed and operated in a specific orientation, thus cannot be understood to limit the present invention.

In addition, terms of “first” and “second” are only for the purpose of description, and cannot be understood to express or imply relative importance or denote implicitly the number of technical features indicated. Therefore, a feature limited by “first” and “second” can include explicitly or implicitly one or more such features. In the description of the present invention, “more” means two or more than two, unless otherwise clearly defined.

In the present invention, unless otherwise clearly specified and defined, terms of “mount”, “connected with”, “connection”, “fix” and the like should be understood in a broad sense, for example, it can be a fixed connection, a detachably connection, or integral; it can be a mechanical connection or an electrical connection; it can be a direct connection, or an indirect connection via an intermediate, or a communication inside two elements or an interactional relation between two elements. For those having ordinary skills in the art, the specific meanings of the above described terms in the present invention can be understood according to specific conditions.

In the present invention, unless otherwise clearly specified and defined, a first feature being “above” or “below” a second feature can include the first feature and the second feature being contacted directly, and also include the first feature and the second feature not being contacted directly but via an additional feature between them. Furthermore, the first feature being “above”, “over” and “on” the second feature may include the first feature being right or ramp above the second feature, or merely represent the first feature being higher than the second feature in horizontal height. The first feature being “below”, “under” and “underneath” the second feature includes the first feature being right or ramp below the second feature, or merely represents the first feature being lower than the second feature in horizontal height.

A breathing machine used in hospitals generally has a respiration valve, functions of which are that: 1. how much flow of gas exhaled by a patient can be detected and fed back to a system to compute; 2. a positive end-expiratory pressure of the respiratory tract of a patient can be set; 3. the respiration valve is closed on inspiratory phase, and opened on respiratory phase. A reliable sealing is required at a diaphragm and a sealing valve port of the respiration valve, freely opening and closing thereof is also required. The processing precision is very high. The sealing performance at the diaphragm and the sealing valve port of the respiration valve in related technologies is always not so good, in this way resulting in distortion in monitoring and calculating of a flow sensor. Furthermore, if the valve port is scratched accidentally in the process of machining, transportation and installation, the entire respiration valve will be scrapped, being not beneficial for saving and maintaining. In addition, a top center of a plastic steam trap and a tray of a waterlogging cup in related technologies will be damaged during a high temperature disinfection process. To this end, the present invention provides a respiration valve.

A respiration valve according to an embodiment of a first aspect of the present invention will be described in detail below with reference to the drawings.

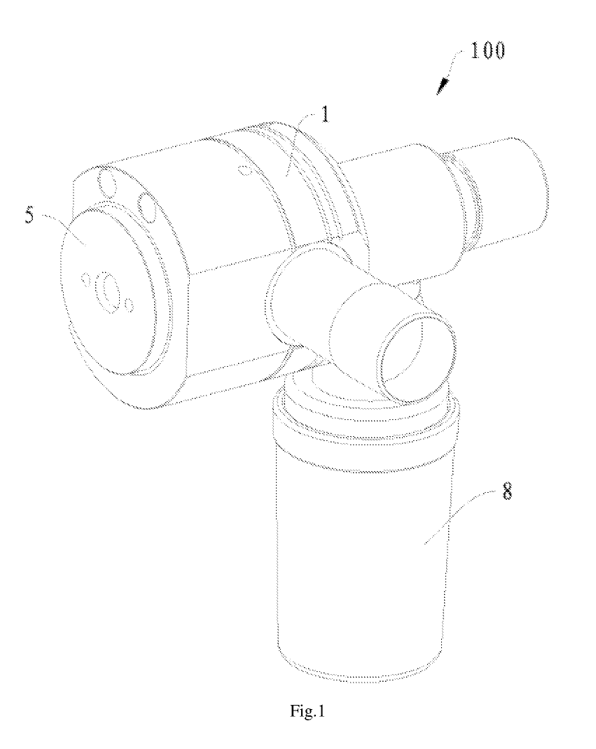

As shown in FIGS. 1 to 3, a respiration valve 100 according to an embodiment of the present invention comprises: a valve body 1, an air resistor 2, a stainless steel valve port 3, a diaphragm 4 and a valve bonnet 5.

Specifically, an air intake channel 101 and air output channel 102 are formed within the valve body 1, the air output channel 102 may be in communication with the air intake channel 101. Moreover, the diaphragm 4 may obstruct between the air intake channel 101 and the air output channel 102, to cause the air intake channel 101 to be not in communication with the air output channel 102. The air intake channel 101 has an air inlet 103 and an air outlet 104, the gas exhaled by a user enters into the respiration valve 100 from the air inlet 103, and may be vented from the respiration valve 100 via the air outlet 104.

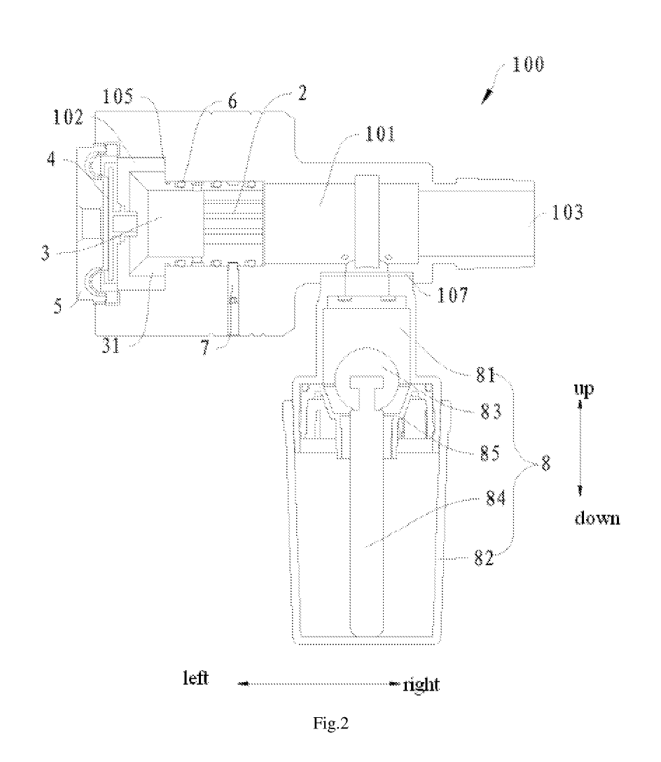

The air resistor 2 is disposed within the air intake channel 101, in order to form pressure difference between both sides of the air resistor 2. The stainless steel valve port 3 is provided at the air outlet 104 of the valve body 1, and one end of the stainless steel valve port 3 (i.e. the end of the stainless steel valve port 3 towards the left as shown in FIG. 2) extends out of the air outlet 104 of the valve body 1. The diaphragm 4 is movably disposed on the valve body 1 between the opened and closed positions of the stainless steel valve port 3. When the diaphragm 4 closes the stainless steel valve port 3, the air intake channel 101 and the air output channel 102 are separated by the diaphragm 4, and the gas that enters into the respiration valve 100 via the air inlet 103 cannot be vented from the respiration valve 100. When the diaphragm 4 opens the stainless steel valve port 3, the air intake channel 101 is in communication with the air output channel 102, and the gas that enters into the respiration valve 100 via the air inlet 103 would be vented from the respiration valve 100, the situation that the diaphragm 4 opens the stainless steel valve port 3 is shown in FIG. 2.

The valve bonnet 5 is disposed on the valve body 1, and cooperates with the valve body 1 to press against an edge of the diaphragm 4, and the valve bonnet 5 is provided with a through hole 501 thereon, the through hole 501 is an inlet for peep gas and can adjust the gas pressure of the space between the air resistor 2 and the diaphragm 4 within the respiration valve 100 needed to open the diaphragm 4.

With the respiration valve 100 according to the embodiment of the present invention, the performance of the sealing of the diaphragm 4 and the stainless steel valve port 3 is improved, an accurate and reliable opening and closing of the valve port 3 in normal working environment is realized, and the tidal volume inhaled and exhaled by the respiration valve 100 may be precisely calculated, the patient may be given with a comfortable breathing mode.

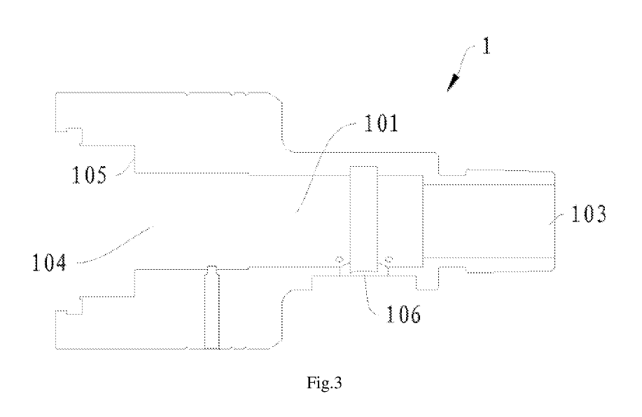

As shown in FIGS. 2 and 3, in some embodiments of the present invention, a step surface 105 is formed at a joint of the air intake channel 101 and the air output channel 102, a part of the stainless steel valve port 3 is disposed within the air intake channel 101, and the other part of the stainless steel valve port 3 extends out of the air outlet 104. A limiting embossment 31 which abuts against the step surface 105 is formed on the outer wall surface of the part of the stainless steel valve port 3 that extends out of the air outlet 104. Thus the stainless steel valve port 3 can be prevented from moving into the air intake channel 101 under the abutment effect of the diaphragm 4.

In some embodiments of the present invention, the air resistor 2 is an aluminium alloy air resistor or a stainless steel air resistor, and the valve body 1 is an aluminium alloy valve body or a stainless steel valve body. Thus the respiration valve 100 has a high-strength structure, facilitates to be produced and assembled, and is rust protective.

Further, in a specific example, the air resistor 2 is made from stainless steel material, and formed integrally with the stainless steel valve port 3. Thus the assembly elements of the respiration valve 100 are decreased, simplifying the assembling process thereof and improving the assembly efficiency thereof.

Advantageously, as shown in FIG. 2, a seal ring 6 is provided between an outer wall surface of the stainless steel valve port 3 and an inner wall surface of the air intake channel 101. Thus the sealing between the stainless steel valve port 3 and the inner wall of the air intake channel 101 is facilitated, avoiding air leakage.

Further, the outer wall surface of the stainless steel valve port 3 is provided with an annular groove, the seal ring 6 is fitted with the annular groove and protrudes beyond the annular groove. Thus the seal ring 3 is located to avoid moving thereof.

Advantageously, the outer wall of the air resistor 2 is provided with a limiting groove, and a jackscrew 7 whose end portion is fitted within the annular groove is provided on the valve body 1. Thus, the air resistor 2 can be located to avoid sliding thereof.

Furthermore, the outer wall of the air resistor 2 can be provided with a plurality of limiting grooves, thus the position of the air resistor 2 can be adjusted according to actual use.

As shown in FIGS. 2 and 3, in some embodiments of the present invention, a bottom of the valve body 1 is provided with a logged water outlet 106 in communication with the air intake channel 101, and a waterlogging cup component 8 in communication with the logged water outlet 106 is provided on the valve body 1. In some embodiments, the waterlogging cup component 8 includes: a waterlogging cup connector 81, a waterlogging cup 82, a seal ball 83 and an ejector rod 84.

Specifically, an upper end of the waterlogging cup connector 81 is connected with the valve body 1, and is in communication with the logged water outlet 106, an annular flange 85 is provided on an inner wall of the waterlogging cup connector 81. The waterlogging cup 82 is detachably connected with a lower end of the waterlogging cup connector 81. The seal ball 83 is disposed within the waterlogging cup connector 81, and located above the annular flange 85. The seal ball 83 can move between a first position and a second position. In some embodiments, the seal ball 83 can abut against the annular flange 85 at the first position to close the waterlogging cup connector 81, and the seal ball 83, when moving upwards, will be located at the second position departing from the first position, and detached from the annular flange 85 to open the waterlogging cup connector 81. The ejector rod 84 can abut against a bottom wall of the waterlogging cup 82 so as to push the seal ball 83 to the second position. In other words, the ejector rod 84 can abut against the bottom wall of the waterlogging cup 82 to eject the seal ball 83 away from the annular flange 85 so as to open the waterlogging cup connector 81. One end of the ejector rod 84 (i.e. the upward end of the ejector rod 84 as shown in FIG. 2) is connected with the seal ball 83, and the other end of the ejector rod 84 extends into the waterlogging cup 82.

When the waterlogging cup 82 is connected with the waterlogging cup connector 81, a lower end of the ejector rod 84 abuts against the bottom wall of the waterlogging cup 82, and an upper end of the ejector rod 82 pushes the seal ball 83 to the second position, thereby causing the waterlogging 82 to be in communication with the air intake channel 101, then logged water within the air intake channel 101 can flow into the waterlogging cup 82. FIG. 2 shows the situation that the seal ball 83 is located at the second position. When the waterlogging cup 82 is detached from the waterlogging cup connector 81, the seal ball 83 will move downwards to abut against the annular flange 85 under the action of gravity, thus sealing the logged water outlet 106 on the valve body 1.

Thus it is easy to clean the water within the air intake channel 101, facilitating the use of the respiration valve 100.

Advantageously, the seal ball 83 is a high temperature resistance seal ball. Therefore, the high temperature resistance seal ball 83 will not be damaged and deformed when high temperature disinfection is performed on the respiration valve 100, such that the seal ball 83 can seal the logged water outlet 106 reliably, avoiding air leakage.

Furthermore, the high temperature resistance seal ball may be a high temperature resistance silicone rubber ball and the like, the ejector rod 84 may be aluminium alloy rod-like. It can be understood to those having ordinary skills in the art that the seal ball 83 may be made from other high temperature resistance materials in prior art.

Further, as shown in FIG. 2, a bottom surface of the valve body 1 is provided with an annular notch 107 surrounding the logged water outlet 106, an upper end of the waterlogging cup connector 81 is fitted within the annular notch 107, and the waterlogging cup connector 81 is connected with the valve body 1 directly via a bolt. Thus it is easy to assemble the respiration valve 100, improving the assembly efficiency and production efficiency of the respiration valve 100.

In the respiration valve 100 according to an embodiment of the present invention, the seal ball 83 is a high temperature resistance silicone rubber ball, the ejector rod 84 is an aluminium alloy ejector rod, avoiding deformation caused by high temperature disinfection which affects the use thereof. When the waterlogging cup is full of water, the waterlogging cup should be taken down to pour out the water in the waterlogging cup, at this time, the rubber ball head will seal the respiration valve under self-weight, the breathing machine can still work normally. A shrink-ring, an air resistor, a sealing gasket, a waterlogging cup tray, a top center of a steam trap and a spring may be omitted in the present invention.

With the respiration valve 100 according to the embodiment of the present invention, the processing performance thereof is improved and the cost thereof is decreased. The stainless steel valve port can be processed separately. It is convenient to manufacture, transport, assemble and maintain. In particular, it is easier to process the parts with satisfactory precision, ensuring the sealing of the diaphragm and valve port.

When the exhaled gas passes through the air resistor 2, a certain pressure difference is created between both ends of the air resistor 2, then a sensor may calculate a flow according to a pipe diameter and the pressure difference. Conditions such as whether a pipeline falls off, or leaks air can be judged. The through hole 501 on the valve bonnet 5 is an inlet for PEEP gas, a certain pressure can be set as desired. When an expiratory pressure is less than the certain pressure, the diaphragm 4 may seal the stainless steel valve port 3. When the pressure of the exhaled gas is larger than the set pressure, the diaphragm 4 is opened by the pressure of the gas, the exhaled gas may be vented out of the respiration valve 100 under the condition that the exhaled gas is kept under a certain pressure, this pressure is the positive end-expiratory pressure of the breathing machine. When turning to an inspiratory phase, a PEEP valve acts on the diaphragm 4 to seal the valve port. The cycle of exhaling and inhaling repeats.

According to the present invention, the performance of the sealing of the diaphragm 4 and the valve port is improved, the accurate and reliable opening and closing of the valve port under a normal working environment is realized, and the tidal volume inhaled and exhaled by the respiration valve can be precisely calculated, and the patient may be given with a comfortable breathing mode and parameters. The structure of the top center of the steam trap is modifed, and the high temperature resistance silicone rubber ball is adopted instead of the top center of the steam trap to perform sealing, which not only simplifies the structure of the respiration valve, but also solves the problem of material deformation caused by high temperature test. The waterlogging cup connector 81 is spirally connected with the valve body 1 directly, instead of connected via an original sealing gasket and 4 screws. The structure of the respiration valve is simplified, and the cost thereof is reduced.

When the respiration valve is used normally, the exhaled gas may be vented only after the air resistor 2 ejects the diaphragm 4. An air channel is hold at two ends of the air resistor 2, a pressure sensor is mounted on the air channel, and may perform precise calculation on the pressure difference generated between a front end and a back end of the air resistor 2, then the current throughput may be calculated. This is the key action of the respiration valve in the breathing machine.

When the gas containing moisture exhaled by the patient passes through the respiration valve 100, the water vapor will be condensed into the waterlogging cup 82. When the water in the waterlogging cup 82 reaches a certain amount, a nurse will take down the waterlogging cup 82 and pour the water out. When the waterlogging cup 82 is taken down, the seal ball 83 and the rod may come into contact with the waterlogging cup connector 81 under the action of gravity to realize a gas sealing, thus the breathing machine will not leak gas, and still can work normally to supply gas to the patient.

The present invention sets a stainless steel valve port 3, and implements the reliable sealing performance, a good processing performance and maintaining performance. Adding a silicone rubber seal ball head will not influence the normal work of the breathing machine during a nurse is working (cleaning liquid of the patient).

The present invention can be implemented on a therapeutic breathing machine. The breathing machine is an assistant ventilation device, and has an inhaling tube and an exhaling tube simultaneously connected to a patient . The patient inhales gas from the inhaling tube of the breathing machine, and then vents gas via the exhaling tube. The respiration valve is one of the most important parts of the breathing machine. A doctor needs to set a positive end-expiratory pressure according to the condition of the patient, and the functions for implementing automated breathing conversion and the like are computed and performed automatically after the valve detects and feeds back, the precise sealing performance and reliability of the respiration valve is the most crucial key to judge whether the quality of the breathing machine is good. How to further improve the measurement accuracy, response time and reliability of the respiration valve has always been an object pursued by the breathing machine industry.

A breathing machine according to an embodiment of a second aspect of the present invention includes the respiration valve 100 according to the first aspect of the present invention.

The breathing machine according to an embodiment of the present invention has the respiration valve 100 according to the above described embodiment of the present invention. The performance of the sealing of the diaphragm 4 and the stainless steel valve port 3 is improved, the accurate and reliable opening and closing of the valve port under normal working environment is realized, and the tidal volume inhaled and exhaled by the respiration valve 100 can be precisely calculated, the patient may be given with a comfortable breathing mode.

The other constituents and operations of the breathing machine according to an embodiment of the present invention are known to those having ordinary skills in the art, no more detail will be described here.

In the description of this specification, the description of reference terms “an embodiment”, “some embodiments”, “example”, “specific example” or “some examples”, etc. means that a specific feature, structure, material or characteristic described in conjunction with the embodiment(s) or example(s) is included in at least one embodiment or example of the present invention. In this specification, the schematic expressions of the above terms need not to be specific for the same embodiment or example. Moreover, the described specific features, structures, materials or characteristics can be combined in any one or more embodiments or examples in a suitable way. In addition, those skilled in the art can join or combine different embodiments or examples described in this specification.

Although the embodiments of the present invention have been showed and described above, it can be understood that, the above embodiments are illustrative, and cannot be understood as a limiting to the present invention. Those having ordinary skills in the art can make changes, modifications, replacements and variations to the above described embodiments within the scope of the present invention.

1. A respiration valve, comprising:
a valve body within which an air intake channel and an air output channel capable of being in communication with the air intake channel are formed, the air intake channel having an air inlet and an air outlet;
an air resistor disposed within the air intake channel;
a stainless steel valve port provided at the air outlet of the valve body, with one end extending out of the air outlet of the valve body;
a diaphragm movably disposed on the valve body between opened and closed positions of the stainless steel valve port; and
a valve bonnet disposed on the valve body, wherein the valve bonnet cooperates with the valve body to press against an edge of the diaphragm, and the valve bonnet is provided with a through hole.

2. The respiration valve according to claim 1, wherein a joint between the air intake channel and the air output channel is formed with a step surface, a part of the stainless steel valve port is disposed within the air intake channel, and the other part of the stainless steel valve port extends out of the air outlet, a limiting embossment is formed on an outer wall surface of the part of the stainless steel valve port which extends out of the air outlet, and the limiting embossment abuts against the step surface.

3. The respiration valve according to claim 1, wherein the air resistor is an aluminum alloy air resistor or a stainless steel air resistor, the valve body is an aluminum alloy valve body or a stainless steel valve body.

4. The respiration valve according to claim 1, wherein the air resistor is formed integrally with the stainless steel valve port.

5. The respiration valve according to claim 1, wherein a seal ring is provided between an outer wall surface of the stainless steel valve port and an inner wall surface of the air intake channel.

6. The respiration valve according to claim 1, wherein a limiting groove is provided on an outer wall of the air resistor, and a jackscrew with an end fitted in the limiting groove is provided on the valve body.

7. The respiration valve according to claim 1, wherein a bottom of the valve body is provided with a logged water outlet in communication with the air intake channel, and a waterlogging cup component in communication with the logged water outlet is provided on the valve body, the waterlogging cup component comprises:
a waterlogging cup connector an upper end of which is connected with the valve body and in communication with the logged water outlet, and an inner wall of which is provided with an annular flange;
a waterlogging cup detachably connected with an lower end of the waterlogging cup connector;
a seal ball movably disposed within the waterlogging cup connector between a first position and a second position, wherein the seal ball abuts against the annular flange to close the waterlogging cup connector at the first position, and moves upwards to depart from the first position to open the waterlogging cup connector at the second position; and
an ejector rod capable of abutting against a bottom wall of the waterlogging cup to push the seal ball to the second position, wherein one end of the ejector rod is connected with the seal ball, and the other end of the ejector rod extends into the waterlogging cup.

8. The respiration valve according to claim 7, wherein the seal ball is a high temperature resistance silicone rubber seal ball.

9. The respiration valve according to claim 7, wherein a bottom surface of the valve body is provided with an annular notch surrounding the logged water outlet, the upper end of the waterlogging cup connector is fitted within the annular notch, and the waterlogging cup connector is connected with the valve body via a bolt directly.

10. A breathing machine comprising a respiration valve comprising:
a valve body within which an air intake channel and an air output channel capable of being in communication with the air intake channel are formed, the air intake channel having an air inlet and an air outlet;
an air resistor disposed within the air intake channel;
a stainless steel valve port provided at the air outlet of the valve body, with one end extending out of the air outlet of the valve body;
a diaphragm movably disposed on the valve body between opened and closed positions of the stainless steel valve port; and
a valve bonnet disposed on the valve body, wherein the valve bonnet cooperates with the valve body to press against an edge of the diaphragm, and the valve bonnet is provided with a through hole.
